(19) United States
(12) Patent Application Publication
(10) Pub. No.: US 2005/0070948 A1
(43) Pub. Date: Mar. 31, 2005

(54) Subcutaneous insertion devices and methods for stimulating subcutaneous regions of patients

(76) Inventor: Andrew E. Kirsteins, Greensboro, NC (US)

Correspondence Address: Brinks Hofer Gilson & Lione;c/o Gregory H. Zayia, NBC Tower, Suite 3600, P.O. Box 10395, Chicago, IL, 60610, US

(21) Appl. No.: 10/672,745

(22) Filed: Sep. 26, 2003

(51) Int. Cl. A61B 17/32

(52) U.S. Cl. 606/189

(57) ABSTRACT

Subcutaneous insertion devices are described that include (a) a needle containing a shaft, a first end, and a second end; and (b) an insulator covering at least a portion of the shaft. The first end of the needle is exposed and is configured for subcutaneous insertion into a patient and an exterior surface of the insulator provides an outermost surface of the shaft. Methods of stimulating subcutaneous regions of a patient using such devices are also described.

### FIELD OF THE INVENTION

The present invention relates generally to devices and methods for subcutaneous therapy and, more particularly, to devices and methods for performing acupuncture.

### BACKGROUND

Acupuncture, one of the oldest therapeutic branches of traditional Chinese medicine, has been practiced for centuries as a method for treating a wide range of afflictions and for inducing anesthesia. Increasingly, acupuncture has gained acceptance throughout western countries as an alternative or supplemental method of medical treatment. While the mechanism by which acupuncture works remains the subject of intensive investigation, the efficacy of the technique for treating numerous maladies and for providing analgesic and/or anesthetic effects is well documented.

Typically, acupuncture involves the insertion of flexible, filiform needles into the skin of a patient at specific regions known as acupoints and at depths sufficient to penetrate certain tissues, musculature or the like. Subsequent manipulation of the needle ends that protrude from the skin (e.g., by manual twisting or vibration) stimulates the subcutaneous tissue and/or intramuscular sensory nerves of the patient.

Electroacupuncture is one variation of traditional acupuncture in which needles are inserted at specific acupoints along the body and then attached with clips to a device that generates electric pulses. The introduction of a mild current through the needles acts as a stimulus on the tissue and/or nerves in the vicinity of the needle.

While adequate stimulation of deep intramuscular sensory nerves is typically needed to produce the desired analgesic effects of acupuncture, pain caused by unintended stimulation of other skin, nerves, tissue or the like often prevents adequate intramuscular sensory nerve stimulation from being achieved, thus limiting the efficacy of the treatment. For example, while intramuscular sensory nerve stimulation in the vicinity of the tip of an acupuncture needle is typically desirable, stimulation of skin, nerves, tissue or the like along the length of the shaft of the needle is typically undesirable inasmuch as it may cause pain and discomfort to a patient. This problem is particularly acute when electric current is applied to the needle since conduction of the current at any portion of the needle except the tip typically causes undesirable stimulation of skin, nerve, tissue or the like.

### SUMMARY

The scope of the present invention is defined solely by the appended claims, and is not affected to any degree by the statements within this summary.

By way of introduction, a subcutaneous insertion device embodying features of the present invention includes (a) a needle containing a shaft, a first end, and a second end; and (b) an insulator covering at least a portion of the shaft. The first end of the needle is exposed and is configured for subcutaneous insertion into a patient and an exterior surface of the insulator provides an outermost surface of the shaft.

A first method of stimulating a subcutaneous region of a patient includes (i) inserting at least a portion of a first subcutaneous insertion device into the patient; and (ii) manipulating the first subcutaneous insertion device, thereby stimulating the subcutaneous region. The first subcutaneous insertion device includes (a) a needle containing a shaft, a first end, and a second end; and (b) an insulator covering at least a portion of the shaft. The first end of the needle is exposed and at least a portion of the first end is inserted into the patient. An exterior surface of the insulator provides an outermost surface of the shaft.

A second method of stimulating a subcutaneous region of a patient includes (i) inserting at least a portion of a first subcutaneous insertion device into the patient at a first acupoint; (ii) inserting at least a portion of a second subcutaneous insertion device into the patient at a second acupoint; and (iii) manipulating at least one of the first and second subcutaneous insertion devices, thereby stimulating a subcutaneous region. Each of the first and the second subcutaneous insertion devices includes (a) a needle containing a shaft, a first end, and a second end; (b) an insulator covering at least a portion of the shaft; and (c) a conductor coupled to an exposed surface of the needle. The first end of the needle is exposed and at least a portion of the first end is inserted into the patient. An exterior surface of the insulator provides an outermost surface of the shaft.

### BRIEF DESCRIPTION OF THE DRAWINGS

FIG. 1 shows a perspective view of a subcutaneous insertion device embodying features of the present invention.

FIG. 2 shows a cross-sectional detailed view taken along the line 1-1 of the subcutaneous insertion device shown in FIG. 1.

### DETAILED DESCRIPTION

Subcutaneous insertion devices have now been discovered that enable stimulation of deep, intramuscular nerves, tissue or the like while minimizing or preventing the undesired stimulation of other sensitive skin, nerves, tissue or the like that come in contact with the device. These devices are particularly useful in performing electroacupuncture, as described below. By way of introduction, the device includes (a) a needle containing a shaft, a first end, and a second end; and (b) an insulator covering at least a portion of the shaft. The first end of the needle is exposed (i.e., is not covered by insulator) and is configured for subcutaneous insertion into a patient and for stimulating deep, intramuscular nerves, tissue or the like. The exterior surface of the insulator corresponds to the outermost surface of the shaft, such that the insulator rather than the needle underneath it contacts all subcutaneous regions brought in contact with the insulator.

In operation, the device is inserted through the skin of a patient beginning with the exposed first end, which is configured for penetrating the surface of the skin (e.g., by having a pointed tip, such as the tip of the needle portion of a safety pin). The device is inserted at a desired point on the skin (e.g., at an acupoint) at a desired angle and to a desired depth in accordance with well-established principles of acupuncture. The exposed first end of the device is positioned in a region of tissue, musculature or the like for which stimulation is desired. Other portions of the device (e.g., the shaft of the needle beneath the surface of the skin) are covered by insulator, which acts to minimize or prevent undesired stimulation of tissue, musculature or the like brought into contact therewith during placement of the device and/or initiation of therapy (e.g., manual manipulation of the device, introduction of an electrical current, etc.). In a presently preferred method of use, the entirety of the exposed first end is positioned beneath the skin of the patient and any skin, nerves, tissue or the like for which stimulation is undesirable are in contact with insulated as opposed to exposed portions of the needle. In this manner, undesired stimulation is minimized or prevented when, for example, an electric current is coupled to the device.

Throughout this description and in the appended claims, the following definitions are to be understood:

The term “stimulating” refers to all manner of affecting a subcutaneous region of a patient with a device, preferably with an acupuncture needle, in an effort to contribute to the attainment of a therapeutic goal.

The phrase “subcutaneous region” refers to any region of a patient located beneath the patient's skin, including tissue, nerve, musculature, blood, bone, blood vessels, arteries, and the like.

The terms “insertion” and “inserting” refer to the piercing of a patient's skin with a device embodying features of the present invention. Although neither term is intended to convey information regarding the extent, depth or angle of insertion, devices used in accordance with the present invention are typically inserted at angles and depths such as may be used with conventional acupuncture needles.

The term “manipulating” refers to all manner of influencing a device that has been inserted subcutaneously into a patient, including but not limited to twisting, vibrating, heating, and connecting the device to a source of electrical current (e.g., with one or more clips). Manipulations of devices embodying features of the present invention may optionally be performed in combination with one or more traditional acupuncture techniques, including but not limited to “cupping” (i.e., stimulating acupoints by applying suction through metal, wood or glass jars, thereby drawing heat from the body), “moxibustion” (i.e., the use of burning herbal agents such as mugwort or wormwood to heat the acupoints), “guasha” (i.e., the use of spoons to apply friction to the skin), “tuina” (i.e., Chinese massage), and the like, and combinations thereof.

The term “acupoints” refers to locations on the surface of the skin that typically lie along meridians or channels of the body as defined by traditional Chinese medicine, which locations have shown utility in conventional acupuncture therapy. As used herein, the phrase “at an acupoint” includes the exact point at which an axis through the center of the acupoint intersects the surface of the skin as well as any region surrounding this point of intersection that is close enough to the acupoint to still prove useful during therapy.

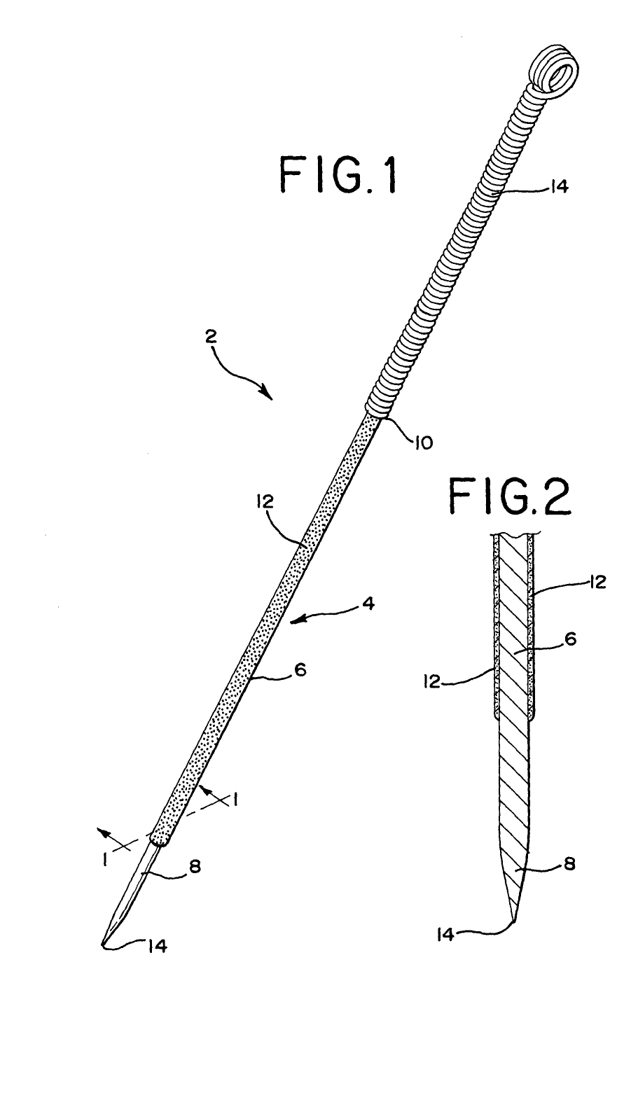

Subcutaneous insertion devices embodying features of the present invention will now be described in reference to the accompanying drawings. FIGS. 1 and 2 show a perspective view and a detailed cross-sectional view, respectively, of a subcutaneous insertion device 2 embodying features of the present invention. Device 2 includes (a) a needle 4 containing a shaft 6, a first end 8, and a second end 10; and (b) an insulator 12 covering at least a portion of the shaft 6. The first end 8 of the needle is exposed and is configured for subcutaneous insertion into a patient. An exterior surface of the insulator 12 provides an outermost surface of the shaft.

All manner of designs and shapes (e.g., conical, cylindrical, beveled, etc.) are contemplated for use in accordance with the point 14 of needle 4. Preferably, the point 14 is conical (e.g., as is the tip of the needle portion of a safety pin) in order to facilitate penetration of the skin. However, the symmetrical V-shaped conical tip pattern illustrated in FIGS. 1 and 2 is to be regarded as strictly illustrative with numerous other symmetrical and unsymmetrical designs being similarly useful.

The needle 4 of subcutaneous insertion devices embodying features of the present invention is preferably solid as is typically the case with conventional acupuncture needles. However, hollow needles or needles containing a central bore that opens to the atmosphere at one or more points along the needle shaft and/or tip have also been contemplated for use in accordance with the present invention. The needle 4 may be manufactured from any material that exhibits the strength, flexibility, and stability to sterilization appropriate for conventional acupuncture needles. Stainless steel is a presently desirable material for use in accordance with the present invention.

All manner of lengths are contemplated for use in accordance with needle 4. Although the length of the needle 4 is not limited, the lengths typically used for conventional acupuncture needles are presently desirable for use in accordance with the present invention. These representative lengths include but are not limited to 0.5″, 1.0″, 1.5″, 2.0″, 2.5″, 3.0″, 3.5″, 4.0″, 4.5″, 5.0″, 5.5″, 6.0″, and the like.

All manner of gauges are contemplated for use in accordance with needle 4. Although the gauge of the needle 4 is not limited, the gauges typically used for conventional acupuncture needles are presently desirable for use in accordance with the present invention. These representative gauges include but are not limited to 28 (0.35 mm), 30 (0.30 mm), 32 (0.25 mm), 34 (0.22 mm), 36 (0.20 mm), 38 (0.18 mm), 40 (0.16 mm), and the like.

The insulator 12 is preferably a biocompatible coating that causes minimal trauma to the subcutaneous regions of the body brought in contact therewith. Presently desirable coatings that may be used in accordance with the present invention include but are not limited to epoxy resins, polyurethanes, polytetrafluoroethylene, silicone, and the like, and combinations thereof. A presently desirable polyurethane resin is that sold under the tradename ESTHANE 5715P by the BF Goodrich Corporation (Charlotte, N.C.).

Polytetrafluoroethylene is a presently preferred coating for use as an insulator in accordance with the present invention. A presently desirable polytetrafluoroethylene is that sold under the tradename TEFLON by the E.I. du Pont de Nemours and Company (Wilmington, Del.). While the thickness of the insulator 12 coated on needle 4 is not limited, it is preferred that the insulator 12 be as thin as possible in order to convey the desired insulating effect without substantially increasing the gauge of the needle 4 underneath the coating. By way of example, it is presently desirable that the thickness of insulator 12 be less than about 50 percent of the overall needle gauge, more desirably less than about 40 percent, more desirably less than about 30 percent, more desirably less than about 20 percent, more desirably less than about 10 percent, and more desirably less than about 5 percent.

The length and/or size of the exposed area on the first end 8 of needle 4 is not limited and may vary according to the type of tissue, musculature or other subcutaneous region to be stimulated. Preferably, the exposed area on the first end 8 is as small as possible in order to convey the desired stimulating effect at the desired subcutaneous region without substantially affecting other subcutaneous regions in the vicinity of needle 4. By way of example, it is presently desirable that the length of the first end 8 is between about one and about forty percent of the overall length of the needle measured from the tip 14 of the first end 8 to the top (not shown) of the second end 10. More desirably, the length of the first end 8 is between about one and about thirty percent, more desirably between about one and about twenty percent, more desirably between about one and about ten percent.

In alternative embodiments in accordance with the present invention, one or more regions of needle 4, such as along shaft 6, may be exposed in addition to the exposed first end 8. In such a manner, a pattern of alternating exposed and insulated regions may be provided along the length of the needle 4. Such patterns may be designed with a view towards stimulating particular layers of the subcutaneous region of the patient without substantially affecting other layers that may lie on either side of a stimulated layer.

Subcutaneous insertion devices embodying features of the present invention are especially desirable for use in electroacupuncture. Accordingly, it is preferred that the subcutaneous insertion devices in accordance with the present invention further include a conductor 14 as shown in FIG. 1. The conductor 14 provides a conduit whereby an electrical impulse from an external electrical source (not shown) may be transferred to the needle 4 and, more particularly, to the exposed first end 8 of the needle 4. Thus, tissue, musculature or the like in the vicinity of the exposed first end 8 will be electrically stimulated. Preferably, the conductor includes any metal, metal alloy, conductive polymer capable of conducting an electrical charge, including but not limited to copper, silver, gold, carbon, platinum, palladium, ceramics, and the like, and combinations thereof. Copper is a presently desirable material for use in accordance with the present invention.

To establish the appropriate electrical connection between the needle 4 and the external electrical source (not shown), the conductor 14 desirably contacts an exposed surface on the needle 4, which is preferably located on a region of the device 2 that does not penetrate the skin of the patient. For example, the second end 10 of needle 4 may contain an exposed surface (i.e., a surface not covered by insulator 12) to which the conductor 14 is coupled. In FIG. 1, the second end 10 is located beneath the conductor 14, which is depicted for purposes of illustration as a coiled metal wire.

A first method of stimulating subcutaneous regions of a patient embodying features of the present invention includes (i) inserting at least a portion of a first subcutaneous insertion device of a type described above into the patient; and (ii) manipulating the first subcutaneous insertion device, thereby stimulating the subcutaneous region.

In certain applications of methods embodying features of the present invention, it is preferred that the subcutaneous insertion device be inserted at an acupoint of the patient. Moreover, as is well understood by those of ordinary skill in the art of acupuncture, the subcutaneous insertion devices embodying features of the present invention may be inserted into the patient at angles ranging from about 1 to about 90 degrees relative to the surface of the skin, more desirably from about 15 to about 90 degrees, more desirably from about 30 to about 90 degrees.

Although the nature of the manipulating is not limited, presently desirable manipulations include but are not limited to twisting, vibrating, heating, and providing an electrical current to the inserted device. The provision of an electrical current to the device is a presently preferred manipulation, as noted above. As is well understood by those of ordinary skill in the art of electroacupuncture, the magnitude of current provided to the device and the duration of the therapy will vary with patient (e.g., depending on overall health, age, etc.) as well as with the type of ailment to be treated. Moreover, the electrical current may be provided in the form of a continuous electrical current and/or as a series of one or more short pulses of electrical current.

Inasmuch as electroacupuncture typically involves the simultaneous use of at least two needles, such that electrical impulses may be passed from one needle to the other, a second method of stimulating subcutaneous regions of a patient includes (i) inserting at least a portion of a first subcutaneous insertion device of a type described above into the patient at a first acupoint; (ii) inserting at least a portion of a second subcutaneous insertion device of a type described above into the patient at a second acupoint; and (iii) manipulating at least one of the first and second subcutaneous insertion devices, thereby stimulating a subcutaneous region.

It is desirable that the first and second subcutaneous insertion devices be inserted at acupoints of a patient and, more desirably, at complementary acupoints of the patient (i.e., a pair of acupoints, which when stimulated with an electrical signal, contribute to the attainment of a desired therapeutic goal).

In accordance with general electroacupuncture recommendations, it is desirable that this therapy not be used on patients with a history of seizures, epilepsy, heart disease or strokes, or on patients with pacemakers. In addition, it is preferred that the first and second subcutaneous insertion devices not be inserted over a patient's head or throat or directly over the patient's heart. Moreover, it is preferred that when the first and second subcutaneous insertion devices are being connected to an electrical current that the current not travel across the midline of the body (i.e., an imaginary line running from the bridge of the nose to the bellybutton).

Devices and methods embodying features of the present invention may be used for inducing anesthesia and in the treatment of a wide variety of ailments including but not limited to all maladies that have previously been addressed by the use of conventional acupuncture and electroacupuncture and conventional acupuncture and electroacupuncture needles. Representative maladies that may be treated in accordance with the present invention include but are not limited to pain, headaches, asthma, nausea, depression, alcohol addiction, drug addiction, nicotine addiction, obesity, arthritis, premenstrual syndrome, fibromyalgia, paralysis, carpal tunnel syndrome, morning sickness, labor pain, schizophrenia, allergy, menopause, infertility, cerebral palsy, multiple sclerosis, and the like, and combinations thereof.

The foregoing detailed description and accompanying drawings have been provided by way of explanation and illustration, and are not intended to limit the scope of the appended claims. Many variations in the presently preferred embodiments illustrated herein will be obvious to one of ordinary skill in the art, and remain within the scope of the appended claims and their equivalents.

1. A subcutaneous insertion device comprising:
a needle comprising a shaft, a first end, and a second end; and
an insulator covering at least a portion of the shaft;
wherein the first end of the needle is exposed and is configured for subcutaneous insertion into a patient; and
wherein an exterior surface of the insulator provides an outermost surface of the shaft.

2. The invention of claim 1 wherein the first end of the needle has a length comprising from about one to about twenty percent of the needle.

3. The invention of claim 1 further comprising a conductor coupled to an exposed surface of the needle.

4. The invention of claim 3 wherein the conductor contacts at least a portion of the second end of the needle.

5. The invention of claim 1 wherein the insulator comprises a biocompatible coating.

6. The invention of claim 5 wherein the coating is selected from the group consisting of an epoxy resin, polyurethane, polytetrafluoroethylene, and combinations thereof.

7. The invention of claim 1 wherein the needle is solid.

8. The invention of claim 1 wherein the needle is hollow.

9. A method of stimulating a subcutaneous region of a patient comprising:
inserting at least a portion of a first subcutaneous insertion device into the patient; and
manipulating the first subcutaneous insertion device, thereby stimulating the subcutaneous region;
wherein the first subcutaneous insertion device comprises:
a needle comprising a shaft, a first end, and a second end; and
an insulator covering at least a portion of the shaft;
wherein the first end of the needle is exposed and wherein at least a portion of the first end is inserted into the patient; and
wherein an exterior surface of the insulator provides an outermost surface of the shaft.

10. The invention of claim 9 wherein the manipulating comprises twisting the first subcutaneous insertion device.

11. The invention of claim 9 wherein the manipulating comprises vibrating the first subcutaneous insertion device.

12. The invention of claim 9 wherein the manipulating comprises heating at least a portion of the first subcutaneous insertion device.

13. The invention of claim 9 wherein the insulator comprises a biocompatible coating.

14. The invention of claim 13 wherein the first subcutaneous insertion device further comprises a conductor coupled to an exposed surface of the needle.

15. The invention of claim 14 wherein the conductor contacts at least a portion of the second end of the needle.

16. The invention of claim 15 wherein the manipulating comprises connecting the conductor to a remote electrical source, such that an electrical impulse may be transferred from the remote electrical source to the first end of the needle.

17. The invention of claim 9 wherein the first subcutaneous insertion device is inserted at an acupoint of the patient.

18. The invention of claim 9 wherein the method is used to treat a malady selected from the group consisting of pain, headaches, asthma, nausea, depression, alcohol addiction, drug addiction, nicotine addiction, obesity, arthritis, premenstrual syndrome, fibromyalgia, paralysis, carpal tunnel syndrome, morning sickness, labor pain, schizophrenia, allergy, menopause, infertility, cerebral palsy, multiple sclerosis, and combinations thereof.

19. The invention of claim 9 wherein the first subcutaneous insertion device is inserted into the patient at an angle of between about 15 and about 90 degrees relative to a surface of skin.

20. The invention of claim 9 wherein all of the first end is inserted into the patient.

21. A method of stimulating a subcutaneous region of a patient comprising:
inserting at least a portion of a first subcutaneous insertion device into the patient at a first acupoint;
inserting at least a portion of a second subcutaneous insertion device into the patient at a second acupoint; and
manipulating at least one of the first and second subcutaneous insertion devices, thereby stimulating a subcutaneous region;
wherein each of the first and the second subcutaneous insertion devices comprises:
a needle comprising a shaft, a first end, and a second end;
an insulator covering at least a portion of the shaft; and
a conductor coupled to an exposed surface of the needle;
wherein the first end of the needle is exposed and wherein at least a portion of the first end is inserted into the patient; and
wherein an exterior surface of the insulator provides an outermost surface of the shaft.

22. The invention of claim 21 wherein the manipulating comprises connecting the conductors of the first and the second subcutaneous insertion devices to a remote electrical source and transferring an electrical impulse from the remote electrical source to the first end of at least one of the first and the second subcutaneous insertion devices.

23. The invention of claim 21 wherein all of the first end is inserted into the patient.

24. The invention of claim 21 wherein the method is used to treat a malady selected from the group consisting of pain, headaches, asthma, nausea, depression, alcohol addiction, drug addiction, nicotine addiction, obesity, arthritis, premenstrual syndrome, fibromyalgia, paralysis, carpal tunnel syndrome, morning sickness, labor pain, schizophrenia, allergy, menopause, infertility, cerebral palsy, multiple sclerosis, and combinations thereof.
